(19) United States
(12) Patent Application Publication
(10) Pub. No.: US 2020/0383887 A1
(43) Pub. Date: Dec. 10, 2020

(54) OXYGENATE SKIN TREATMENT SYSTEM

(71) Applicant: L'OREAL, Paris (FR)

(72) Inventors: Anne-Laure Suzanne BERNARD, New York, NY (US); Rebecca Chen, Princeton, NJ (US); Zhi Pan, Ridgewood, NJ (US)

(21) Appl. No.: 16/431,288

(22) Filed: Jun. 4, 2019

(51) Int. Cl. A61K 8/22; A61K 8/85; A61Q 19/08; A61K 8/92; A61K 8/25; A61K 8/43; A61K 8/44; A61K 8/24; A61K 8/04; A61K 8/06; A61Q 19/02

(52) U.S. Cl. CPC A61K 8/22; A61K 8/85; A61Q 19/08; A61K 8/92; A61K 8/25; A61K 8/43; A61K 2800/30; A61K 8/24; A61K 8/042; A61K 8/06; A61Q 19/02; A61K 2800/413; A61K 2800/882; A61K 8/44

(57) ABSTRACT

A skin treatment system is disclosed including an oxygenate composition having at least one peroxide in a first aqueous phase and an activator composition, the activator composition including at least one alkaline booster in a second aqueous phase and at least one water-soluble peroxide decomposition catalyst in the second aqueous phase. Intermixing the oxygenate composition and the activator composition at least partially decomposes the at least one peroxide, liberating O₂, and the skin treatment system in contact with keratinous tissue induces reduced irritation to the keratinous tissue relative to a comparative skin treatment system which is identical to the skin treatment system except for lacking the at least one water-soluble peroxide decomposition catalyst.

### FIELD OF TECHNOLOGY

The present disclosure is directed to oxygenate skin treatment systems. More specifically, the present disclosure is directed to oxygenate skin treatment systems including an activator composition.

### BACKGROUND

Oxygen therapy has drawn significant attention in recent years for uses such as skin-brightening, skin repair, and wound healing. Lack of oxygen in wounded areas may result in poor collagen deposition and a prolonged healing process. Oxygen, of course, is present in air and is taken into the human body via respiration, and such respiratory oxygen must dissolve into bodily fluid to circulate into tissues to support cellular activities. Supplemental oxygen may be delivered from the external environment via diffusion through the skin barrier (hyperbaric oxygen therapy), however, due to the strong barrier function of skin and the conversion of oxygen from the gaseous to dissolved state, the delivery efficiency is relatively low. Additional challenges arise for hyperbaric oxygen therapy, in that some patients cannot tolerate the side effects of the treatments, afford the treatments, or gain access to a hyperbaric chamber.

Topical delivery of dissolved oxygen is another approach. Dissolved oxygen may be delivered to a skin surface via the use of perfluorocarbons as oxygen carriers or through chemical degradation of peroxide compounds to generate oxygen gas. A burst treatment of supplemental oxygen may inhibit proliferation of anaerobic bacteria, accelerate fibroplasia and epithelialization. Typically, such products employ oxidizing agents such as hydrogen peroxide. However, contact with hydrogen peroxide in such products may, under certain conditions, produce mild to moderate skin irritation and eye tearing.

U.S. Patent Application Publication No. 2014/0294944 A1 to Schumacher discloses a composition for the delivery of oxygen having microencapsulated peroxide and microencapsulated catalyst that liberate oxygen upon sufficient contact with each. However, accomplishing the microencapsulation of hydrogen peroxide as disclosed in this publication is both difficult and complicated.

U.S. Pat. No. 9,181,093 B2 to Karandikar et al., discloses a two-part spray for the liberation and delivery of oxygen through the use of a first part that is a peroxide-containing solution and a second part that is a nanoparticle manganese dioxide catalyst, which, when mixed together, provide oxygen. However, the manganese dioxide nanoparticles utilized in this publication are not water-soluble which complicates delivery of the composition. Moreover, due to the encapsulation, the reaction efficiency would well be sacrificed, and the oxygen release could be impeded. Further, there may be respiratory health concerns with encapsulated particles.

It is an object of the present invention to provide an oxygenate skin treatment system that overcomes at least one of the aforementioned drawbacks.

### BRIEF SUMMARY

The summary is provided to introduce a selection of concepts in a simplified form that are further described below in the detailed description of the invention. This summary is not intended to identify key features of the claimed subject matter, nor is it intended to be used as an aid in determining the scope of the claimed subject matter.

In an exemplary embodiment, a skin treatment system includes an oxygenate composition having at least one peroxide in a first aqueous phase and an activator composition, the activator composition including at least one alkaline booster in a second aqueous phase and at least one water-soluble peroxide decomposition catalyst in the second aqueous phase. Intermixing the oxygenate composition and the activator composition at least partially decomposes the at least one peroxide, liberating 02, and the skin treatment system in contact with keratinous tissue induces reduced irritation to the keratinous tissue relative to a comparative skin treatment system which is identical to the skin treatment system except for lacking the at least one water-soluble peroxide decomposition catalyst.

In another exemplary embodiment, a skin treatment system, includes an oxygenate composition including at least one peroxide in a first aqueous phase, an activator composition, and at least one fatty compound in at least one of the oxygenate composition and the activator composition. The activator composition includes at least one alkaline booster in a second aqueous phase and at least one water-soluble peroxide decomposition catalyst in the second aqueous phase. Generally, non-limiting examples of alkaline agents include alkali metal hydroxides (e.g., lithium hydroxide, sodium hydroxide, potassium hydroxide, magnesium hydroxide), alkaline-earth metal hydroxides (e.g., calcium hydroxide), alkali metal silicates, (e.g., sodium silicate, lithium silicate, and potassium silicate), alkali metal carbonates (e.g., lithium carbonate, sodium carbonate, potassium carbonate), alkaline-earth metal carbonates (e.g., calcium carbonate), organic carbonates (e.g., guanidine carbonate), basic amino acids (arginine, lysine, histidine), and their polymers (poly arginine, poly lysine, etc.), organic amines, such as alkanolamines (e.g., monoethanolamine, diethanolamine, triethanolamine, aminomethyl propanol), ammonium hydroxide, and a mixture thereof. A more exhaustive but non-limiting description of alkaline agents that may be included in the alkaline compositions is provided later, under the heading “Alkaline Agents.” The at least one alkaline booster is selected from the group consisting of guanidine carbonate, arginine, monoethanolamine, triethanolamine, potassium hydroxide, sodium bicarbonate, sodium silicate, montmorillonite, and combinations thereof. The at least one water-soluble peroxide decomposition catalyst is selected from the group consisting of a manganese salt of pyrrolidone carboxylic acid, manganese gluconate, manganese sulfate, manganese chloride, a copper salt of pyrrolidone carboxylic acid, copper gluconate, copper sulfate, copper chloride, a zinc salt of pyrrolidone carboxylic acid, zinc gluconate, zinc sulfate, zinc chloride, and combinations thereof. The at least one fatty compound is selected from the group consisting of liquid petroleum jelly, polydecenes, fatty acids, liquid esters of fatty acids, fatty alcohols, and combinations thereof. Intermixing the oxygenate composition and the activator composition at least partially decomposes the at least one peroxide, liberating O₂. The skin treatment system in contact with keratinous tissue induces reduced irritation to the keratinous tissue relative to a comparative skin treatment system which is identical to the skin treatment system except for lacking the at least one water-soluble peroxide decomposition catalyst. Intermixing the oxygenate composition and the activator composition produces reduced reactive oxygen species relative to the comparative skin treatment system. Intermixing the oxygenate composition and the activator composition liberates O₂ at an increased rate relative to the comparative skin treatment system. Upon intermixing of the oxygenate composition and the activator composition, the skin treatment system includes a form selected from the group consisting of an emulsion, a gel, a solution, and combinations thereof. In some embodiments, the composition includes at least one fatty compound. In some particular embodiments, the at least one fatty compound constitutes at least 30 wt % of the total weight of the skin treatment system. The at least one alkaline booster constitutes from 0.5 wt % to 5.5 wt % of the total weight of the skin treatment system. The at least one water-soluble peroxide decomposition catalyst constitutes from 0.1 wt % to 6 wt % of the total weight of the skin treatment system. The at least one peroxide constitutes from 0.5 wt % to 5 wt % of the total weight of the skin treatment system.

Other features and advantages of the present disclosure will be apparent from the following more detailed description of the preferred embodiment which illustrates, by way of example, the principles of the disclosure.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DETAILED DESCRIPTION

The following examples are intended to further illustrate the present disclosure. They are not intended to limit the disclosure in any way. Unless otherwise indicated, all parts are by weight.

The term “alkaline booster” as used herein means compositions comprising at least one alkaline agent with a final pH >7, or a final pH >8. The alkaline agent may be organic or mineral or hybrid with a pKa at 25° C. greater than 7.5.

The terms “ammonia-free” and “persulfate-free” means that the composition comprises less than a trace amount, for example, less than 0.001% by weight of the composition, of an ammonia containing ingredient or a persulfate containing ingredient, and more particularly that the ingredient has not been intentionally added, but may be included as a by-product or carry-over of another ingredient. In some embodiments, each of ammonia-free and persulfate-free means that the composition is devoid, respectively, of ammonia and ammonia containing ingredients, and of persulfates, persulfate containing ingredients, perborates, alkali metal percarbonates, alkaline-earth metal percarbonates, peracids and precursors thereof.

“Cosmetically acceptable” means compatible with any keratinous substrate. For example, “cosmetically acceptable carrier” means a carrier that is compatible with any keratinous substrate.

“Skin-brightening” means and refers to improvement in radiance, glow effect and lightening effect to keratinous tissue. Skin brightness is related to luminosity and can be measured instrumentally by the change of luminosity (ΔL).

“Mechanical exfoliation agent” means a particulate material with a size range of from about 25, preferably from about 50 to about 500, preferably about 100 to 250 micrometers. It could be powders from ground apricot seeds, crushed walnut shells, coconut shells, almond seeds and shells and sawdust, various solid polymer powders, and various inorganic particles such as sand, salt, alumina, silica, alumino-silicates, lava stones, various phosphates, borates, sulfates and carbonates.

Applicants have formulated a composition including at least one peroxide in a first aqueous phase and at least one alkaline booster and at least one water-soluble peroxide decomposition catalyst in a second aqueous phase. Unexpectedly, the composition demonstrates beneficial effects relating to skin-brightening, skin repair, and/or wound healing while also inducing reduced irritation to the keratinous tissue relative to an otherwise identical treatment system lacking the at least one water-soluble peroxide decomposition catalyst.

In one embodiment, a skin treatment system includes an oxygenate composition and an activator composition. The oxygenate composition includes at least one peroxide in a first aqueous phase. The activator composition includes at least one alkaline booster in a second aqueous phase and at least one water-soluble peroxide decomposition catalyst in the second aqueous phase. Intermixing the oxygenate composition and the activator composition at least partially decomposes the at least one peroxide, liberating O₂. The skin treatment system in contact with keratinous tissue induces reduced irritation to the keratinous tissue relative to a comparative skin treatment system which is identical to the skin treatment system except for lacking the at least one water-soluble peroxide decomposition catalyst. In a further embodiment, intermixing the oxygenate composition and the activator composition liberates O₂ at an increased rate relative to the comparative skin treatment system.

Upon intermixing of the oxygenate composition and the activator composition, the skin treatment system may be an emulsion, a gel, or a solution.

In one embodiment, the skin treatment system is free of ammonia and persulfate. In another embodiment, the skin treatment system is free of encapsulating microspheres. In a further embodiment, the skin treatment system is free of ammonia, persulfate, and encapsulating microspheres.

Peroxide

The at least one peroxide may be any suitable peroxide, including, but not limited to, hydrogen peroxide, urea peroxide, carbamide peroxide, PVP hydrogen peroxide, or combinations thereof.

In one embodiment, the at least one peroxide constitutes from 0.1 wt % to 5 wt % of the total weight of the skin treatment system, alternatively from 0.1 wt % to 0.5 wt %, alternatively from 0.5 wt % to 1 wt %, alternatively from 1 wt % to 1.5 wt %, alternatively from 1.5 wt % to 2 wt %, alternatively from 2 wt % to 2.5 wt %, alternatively from 2.5 wt % to 3 wt %, alternatively from 3 wt % to 3.5 wt %, alternatively from 3.5 wt % to 4 wt %, alternatively from 4 wt % to 4.5 wt %, alternatively from 4.5 wt % to 5 wt %, or any suitable combination, sub-combination, range, or sub-range thereof by weight, based on the weight of the skin treatment system. One of ordinary skill in the art, however, will appreciate that other ranges are within the scope of the invention.

Alkaline Booster

The at least one alkaline booster may be any suitable booster composition. In one embodiment, suitable booster compositions include, but are not limited to, organic amines and salts, mineral based salts, alkali metal hydroxides, alkali earth metal hydroxides, alkali metal salts, clays, or combinations thereof. In a further embodiment, suitable booster compositions include, but are not limited to, guanidine carbonate, arginine, monoethanolamine, triethanolamine, potassium hydroxide, sodium bicarbonate, sodium silicate, montmorillonite, or combinations thereof. Although these alkaline boosters are given as an example, it will be appreciated that other alkaline boosters compatible with cosmetic applications known in the art may be used. Thus, the alkaline booster may be organic or mineral or hybrid with a pKa at 25° C. greater than 7.5, for example, lysine, carnosine, and NaOH.

The at least one alkaline booster may constitute any suitable proportion of the total weight of the skin treatment system, including, but not limited to, from 0.5 wt % to 10 wt %, alternatively from 0.5 wt % to 1.5 wt %, alternatively from 1 wt % to 2 wt %, alternatively from 1.5 wt % to 2.5 wt %, alternatively from 2 wt % to 3 wt %, alternatively from 2.5 wt % to 3.5 wt %, alternatively from 3 wt % to 4 wt %, alternatively from 3.5 wt % to 4.5 wt %, alternatively from 4 wt % to 5 wt %, alternatively from 4.5 wt % to 5.5 wt %, or any suitable combination, sub-combination, range, or sub-range thereof by weight, based on the weight of the skin treatment system. One of ordinary skill in the art, however, will appreciate that other ranges are within the scope of the invention.

Peroxide Decomposition Catalyst

The at least one water-soluble peroxide decomposition catalyst may be any suitable catalyst, including, but not limited to, a metal salt. Suitable metal salts include, but are not limited to, a manganese salt of pyrrolidone carboxylic acid, manganese gluconate, manganese sulfate, manganese chloride, a copper salt of pyrrolidone carboxylic acid, copper gluconate, copper sulfate, copper chloride, a zinc salt of pyrrolidone carboxylic acid, zinc gluconate, zinc sulfate, zinc chloride, or combinations thereof.

In one embodiment the at least one water-soluble peroxide decomposition catalyst constitutes from 0.1 wt % to 6 wt % of the total weight of the skin treatment system, alternatively from 0.1 wt % to 1 wt %, alternatively from 0.5 wt % to 1.5 wt %, alternatively from 1 wt % to 2 wt %, alternatively from 1.5 wt % to 2.5 wt %, alternatively from 2 wt % to 3 wt %, alternatively from 2.5 wt % to 3.5 wt %, alternatively from 3 wt % to 4 wt %, alternatively from 3.5 wt % to 4.5 wt %, alternatively from 4 wt % to 5 wt %, alternatively from 4.5 wt % to 5.5 wt %, alternatively from 5 wt % to 6 wt %, or any suitable combination, sub-combination, range, or sub-range thereof by weight, based on the weight of the skin treatment system. One of ordinary skill in the art, however, will appreciate that other ranges are within the scope of the invention.

Water

The skin treatment system may include any suitable amount of water, including, but not limited to, from 10 wt % to 50 wt %, alternatively from 10 wt % to 20 wt %, alternatively from 15 wt % to 25 wt %, alternatively from 20 wt % to 30 wt %, alternatively from 25 wt % to 35 wt %, alternatively from 30 wt % to 40 wt %, alternatively from 35 wt % to 45 wt %, alternatively from 40 wt % to 50 wt %, or any suitable combination, sub-combination, range, or sub-range thereof by weight, based on the weight of the skin treatment system. One of ordinary skill in the art, however, will appreciate that other ranges are within the scope of the invention.

Fatty Compound

In some embodiments, at least one of the oxygenate composition and the activator composition further includes at least one optional fatty compound. The at least one fatty compound may include any suitable composition, including, but not limited to, liquid petroleum jelly, polydecenes, fatty acids, liquid esters of fatty acids, fatty alcohols, or combinations thereof.

In one embodiment, the at least one fatty compound constitutes at least 30 wt % of the total weight of the skin treatment system, alternatively at least 35 wt % of the total weight of the skin treatment system, alternatively at least 40 wt % of the total weight of the skin treatment system, alternatively at least 45 wt % of the total weight of the skin treatment system, alternatively at least 50 wt % of the total weight of the skin treatment system, alternatively from 30 wt % to 70 wt % of the total weight of the skin treatment system, alternatively from 30 wt % to 40 wt % of the total weight of the skin treatment system, alternatively from 35 wt % to 45 wt % of the total weight of the skin treatment system, alternatively from 40 wt % to 50 wt % of the total weight of the skin treatment system, alternatively from 45 wt % to 55 wt % of the total weight of the skin treatment system, alternatively from 50 wt % to 60 wt %, alternatively from 55 wt % to 65 wt %, alternatively from 60 wt % to 70 wt % of the total weight of the skin treatment system, or any suitable combination, sub-combination, range, or sub-range thereof by weight, based on the weight of the skin treatment system. One of ordinary skill in the art, however, will appreciate that other ranges are within the scope of the invention.

Surfactant

The skin treatment system may include at least one surfactant. Suitable surfactants include, but are not limited to, nonionic surfactants, anionic surfactants, or combinations thereof. In some embodiments, the skin treatment system includes one, two, three, or more surfactants.

Exemplary anionic surfactants include, but are not limited to, the salts (in particular alkali metal salts, for example, sodium salts, amine salts such as aminoalcohol salts or alkaline-earth metal salts such as magnesium salts) of the following compounds: alkyl sulfates, alkyl ether sulfates, alkylamido ether sulfates, alkylaryl polyether sulfates, monoglyceride sulfates; alkyl sulfonates, alkylamide sulfonates, alkylaryl sulfonates, α-olefin sulfonates, paraffin sulfonates; alkyl phosphates, alkyl ether phosphates; alkylsulfosuccinates, alkyl ether sulfosuccinates, alkyl amidesulfosuccinates; alkyl sulfosuccinamates; alkyl sulfoacetates; acylsarcosinates; acylisethionates and N-acyltaurates; salts of fatty acids such as oleic acid, ricinoleic acid, palmitic acid or stearic acid, coconut oil acid or hydrogenated coconut oil acid; alkyl-D-galactoside uronic acid salts; acyllactylates; salts of polyoxyalkylenated alkyl ether carboxylic acids, of polyoxyalkylenated alkylaryl ether carboxylic acids or of polyoxyalkylenated alkylamido ether carboxylic acids, in particular those having from 2 to 50 ethylene oxide groups; and mixtures thereof.

Exemplary nonionic surfactants include, but are not limited to, monooxyalkylenated or polyoxyalkylenated, monoglycerolated or polyglycerolated nonionic surfactants. The oxyalkylene units may be oxyethylene or oxypropylene units, or a combination thereof. In some embodiments, the oxyalkylene units are oxyethylene units. Exemplary oxyalkylenated nonionic surfactants include, but are not limited to: oxyalkylenated (C₈-C₂₄)alkylphenols, saturated or unsaturated, linear or branched, oxyalkylenated C₈-C₃₀ alcohols, saturated or unsaturated, linear or branched, oxyalkylenated C₈-C₃₀ amides, esters of saturated or unsaturated, linear or branched, C₈-C₃₀ acids and of polyethylene glycols, polyoxyethylenated esters of saturated or unsaturated, linear or branched, C₈-C₃₀ acids and of sorbitol, saturated or unsaturated, oxyethylenated plant oils, condensates of ethylene oxide and/or of propylene oxide, and mixtures thereof. In one embodiment, the at least one surfactant selected from Ceteth-2, decyl glucoside, Steareth-2, Steareth-20, and combinations thereof.

In one embodiment, the at least one surfactant constitutes from 0.1 wt % to 15 wt % of the total weight of the skin treatment system, alternatively from 0.1 wt % to 10 wt %, alternatively from 5 wt % to 15 wt %, alternatively from 0.1 wt % to 5 wt %, alternatively from 2.5 wt % to 7.5 wt %, alternatively from 5 wt % to 10 wt %, alternatively from 7.5 wt % to 12.5 wt %, alternatively from 10 wt % to 15 wt %, or any suitable combination, sub-combination, range, or sub-range thereof by weight, based on the weight of the skin treatment system. One of ordinary skill in the art, however, will appreciate that other ranges are within the scope of the invention.

Polymer

The skin treatment system may include one or more polymers. Suitable polymers include, but are not limited to, cellulose gums, for example, cetyl hydroxyethyl cellulose, and sclerotium gum, and combinations thereof.

In one embodiment, the one or more polymers constitute from 0.1 wt % to 5 wt % of the total weight of the skin treatment system, alternatively from 0.1 wt % to 1 wt %, alternatively from 0.5 wt % to 1.5 wt %, alternatively from 1 wt % to 2 wt %, alternatively from 1.5 wt % to 2.5 wt %, alternatively from 2 wt % to 3 wt %, alternatively from 2.5 wt % to 3.5 wt %, alternatively from 3 wt % to 4 wt %, alternatively from 3.5 wt % to 4.5 wt %, alternatively from 4 wt % to 5 wt %, or any suitable combination, sub-combination, range, or sub-range thereof by weight, based on the weight of the skin treatment system.

Chelating Agents

The skin treatment system may include one or more chelating agents. Suitable chelating agents include, but are not limited to, ethylenediaminetetraacetic acid (EDTA), tetrasodium etidronate, tetrasodium pyrophosphate, pentasodium ethylenediamine tetramethylene phosphonate, sodium staninate, and combinations of these.

In one embodiment, the one or more chelating agents constitute from 0.01 wt % to 2 wt % of the total weight of the skin treatment system, alternatively from 0.01 wt % to 0.4 wt %, alternatively from 0.2 wt % to 0.6 wt %, alternatively from 0.4 wt % to 0.8 wt %, alternatively from 0.6 wt % to 1 wt %, alternatively from 0.8 wt % to 1.2 wt %, alternatively from 1 wt % to 1.4 wt %, alternatively from 1.2 wt % to 1.6 wt %, alternatively from 1.4 wt % to 1.8 wt %, alternatively from 1.6 wt % to 2 wt %, or any suitable combination, sub-combination, range, or sub-range thereof by weight, based on the weight of the skin treatment system.

Optional Components

The skin treatment system may include one or more actives. Exemplary suitable actives include, but are not limited to: humectants, such as acetamide MEA, glycols, such as glycerin and propylene glycol; alcohols; anti-microbial components; salicylic acid; citric acid; alpha hydroxy acid; phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenol s, betacyanins, capsacinoids, hydroxybenzoketones, methoxyphenols, naphthoquinones, and phenolic terpenes; resveratrol; curcumin; pinoresinol; ferulic acid; hydroxytyrosol; cinnamic acid; caffeic acid; p-coumaric acid; baicalin (Scutellaria Baicalensis root extract); pine bark extract (Pinus Pinaster bark/bud extract); ellagic acid; vitamins and vitamin derivatives, such as tocopherol and ascorbic acid; and combinations thereof.

The skin treatment system may include one or more additional components. Exemplary suitable additional components include, but are not limited to: fillers such as clays, talc, organic thickeners with for instance, anionic, cationic, nonionic, and amphoteric polymeric associative thickeners and combinations thereof; penetrants; sequestrants; fragrances; dispersants; film-forming agents; ceramides; opacifiers, and combinations thereof.

In one embodiment, the one or more actives and additional components constitute from 0 wt % to 50 wt % of the total weight of the skin treatment system, alternatively from 0.01 wt % to 50 wt %, alternatively from 0.1 wt % to 10 wt %, alternatively from 5 wt % to 15 wt %, alternatively from 10 wt % to 20 wt %, alternatively from 15 wt % to 25 wt %, alternatively from 20 wt % to 30 wt %, alternatively from 25 wt % to 35 wt %, alternatively from 30 wt % to 40 wt %, alternatively from 35 wt % to 45 wt %, alternatively from 40 wt % to 50 wt %, or any suitable combination, sub-combination, range, or sub-range thereof by weight, based on the weight of the skin treatment system.

In one embodiment, the one or more actives constitute from 0.01 wt % to 3 wt % of the total weight of the skin treatment system, alternatively from 0.01 wt % to 0.4 wt %, alternatively from 0.2 wt % to 0.6 wt %, alternatively from 0.4 wt % to 0.8 wt %, alternatively from 0.6 wt % to 1 wt %, alternatively from 0.8 wt % to 1.2 wt %, alternatively from 1 wt % to 1.4 wt %, alternatively from 1.2 wt % to 1.6 wt %, alternatively from 1.4 wt % to 1.8 wt %, alternatively from 1.6 wt % to 2 wt %, alternatively from 1.8 wt % to 2.2 wt %, alternatively from 2 wt % to 2.4 wt %, alternatively from 2.2 wt % to 2.6 wt %, alternatively from 2.4 wt % to 2.8 wt %, alternatively from 2.6 wt % to 3 wt %, or any suitable combination, sub-combination, range, or sub-range thereof by weight, based on the weight of the skin treatment system.

The skin treatment system may include one or more preservatives. Exemplary suitable preservatives include, but are not limited to, sodium salicylate, sodium citrate.

The skin treatment system may include one or more vitamins. Exemplary suitable vitamins include, but are not limited to, ascorbic acid, tocopherol, and combinations thereof.

In one embodiment, the one or more preservatives and vitamins constitute from 0.01 wt % to 10 wt % of the total weight of the skin treatment system, alternatively from 0.01 wt % to 2 wt %, alternatively from 1 wt % to 3 wt %, alternatively from 2 wt % to 4 wt %, alternatively from 3 wt % to 5 wt %, alternatively from 4 wt % to 6 wt %, alternatively from 5 wt % to 7 wt %, alternatively from 6 wt % to 8 wt %, alternatively from 7 wt % to 9 wt %, alternatively from 8 wt % to 10 wt %, or any suitable combination, sub-combination, range, or sub-range thereof by weight, based on the weight of the skin treatment system.

Articles of Manufacture

The skin treatment system may be provided in a kit or other article of manufacture, such as, a mask, a cream, or a lotion. In one embodiment, the article of manufacture may be a packet with separate packages or chambers separated by a frangible seal between the chambers. In use, the seal is broken by the user to contact the separately packaged oxygenate composition and activator composition, and mix them prior to application onto the skin.

The packaging may be a single tube with a seal between the two components that is broken to mix the oxygenate composition and the activator composition. Alternatively, the packaging is otherwise sufficient to retain the oxygenate composition separate from the activator composition until the composition is intended to be applied to keratinous tissue.

It will be appreciated that in the various embodiments of packaging, that the oxygenate composition and the activator composition that are packaged are maintained as separate until the composition is intended to be applied to keratinous tissue, at which time they are combined and mixed. Variously, each of the oxygenate composition and the activator composition may include any one or more of the fatty compound and any one or more of water, surfactants, polymers, actives and additional components. Further, any one or more of the fatty compound and any one or more of water, surfactants, polymers, actives and additional components may be separately packaged from the oxygenate composition and the activator composition, such that the article of manufacture may comprise more than two separate packages or package chambers.

In accordance with the various embodiments, the skin treatment system is in a form including a suspension, lotion, cream, serum, essence, gel, stick, spray, ointment, paste, foam, mousse, cream, wipe, patch, strip, film-forming product, facial masks, or skin masks.

The oxygenate composition and the activator composition may be packaged in isolation from one another.

### Examples

Table 1 discloses exemplary inventive formulations two (Examples 1 and 2) and four comparative formulations (Examples 3-5). In an in vitro experiment, the skin treatment systems of Examples 1-5 were applied to keratinous tissue. Each of Examples 1, 2, and 5 have the same amounts of manganese on a molar basis. Skin Brightness was determined using instrumental evaluation. Skin Brightness is related to luminosity and can be measured instrumentally by the change of luminosity (ΔL).

TABLE 1
Inventive and Comparative Formulations

| | | | Ex. 3 | Ex. 4 | Ex. 5 |
|---|---|---|---|---|---|
| INCI | Ex. 1 | Ex. 2 | (comparative) | (comparative) | (comparative) |
| INGREDIENT | (wt %) | (wt %) | (wt %) | (wt %) | (wt %) |
| Oxygenate | | | | | |
| Composition | | | | | |
| Hydrogen Peroxide | 3.5 | 3.5 | 3.5 | 3.5 | 3.5 |
| Mineral Oil | 50 | 50 | 50 | 50 | 50 |
| Activator | | | | | |
| Composition | | | | | |
| Arginine | 4 | 4 | | 4 | 4 |
| Manganese Oxide | | | | | 0.06 |
| Manganese | 0.3 | | | | |
| Gluconate | | | | | |
| Manganese PCA | | 0.21 | | | |
| Mineral Oil | 50 | 50 | | 50 | 50 |
| ΔL | 2.6 | 2.5 | 0.6 | 1.9 | 1.2 |
| Solubilized oxygen | 40 | 38.7 | 6.4 | 7 | 7.7 |
| level in formulation | | | | | |
| (mg/L) (1 minute | | | | | |
| after mixing) | | | | | |
| Solubilized oxygen | 35 | 40.7 | 3.9 | 4.2 | 5.7 |
| level in formulation | | | | | |
| (mg/L) (15 minute | | | | | |
| after mixing) | | | | | |

* Solubilized oxygen level in air is ~9.3 mg/L.

Table 2 discloses exemplary inventive formulations in which the oxygenate composition and the activator composition have been intermixed, each of which satisfies the standards for a skin treatment system.

TABLE 2
Inventive Formulations

| | Example 6 | Example 7 |
|---|---|---|
| INCI INGREDIENT | (wt %) | (wt %) |
| Manganese PCA | 0.5 | |
| Tetrasodium Etidronate | 0.03 | 0.03 |
| EDTA | 0.1 | 0.1 |
| Manganese Gluconate | | 0.15 |
| Arginine | 2 | 2 |
| Hydrogen Peroxide | 2 | 2 |
| Tetrasodium Pyrophosphate | 0.02 | 0.02 |
| Water | 34.1175 | 34.4675 |
| Mineral Oil | 56.75 | 56.75 |
| Surfactants | 3.53 | 3.53 |
| Optional Actives | 0.1775 | 0.1775 |
| Polymer | 0.775 | 0.775 |

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on.

All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated. As used herein, “about”, unless otherwise indicated, encompasses the expected variation in quantification inherent in the typical measurement system applied thereto as well as ordinary experimental variance, as would be understood and expected by a person having ordinary skill in the art.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While the disclosure has been described with reference to a preferred embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A skin treatment system, comprising:
an oxygenate composition including at least one peroxide in a first aqueous phase; and
an activator composition including:
at least one alkaline booster in a second aqueous phase; and
at least one water-soluble peroxide decomposition catalyst in the second aqueous phase,
wherein intermixing the oxygenate composition and the activator composition at least partially decomposes the at least one peroxide, liberating O₂, and
wherein the skin treatment system in contact with keratinous tissue induces reduced irritation to the keratinous tissue relative to a comparative skin treatment system which is identical to the skin treatment system except for lacking the at least one water-soluble peroxide decomposition catalyst.

2. The skin treatment system of claim 1, wherein intermixing the oxygenate composition and the activator composition liberates O₂ at an increased rate relative to the comparative skin treatment system.

3. The skin treatment system of claim 1, wherein upon intermixing of the oxygenate composition and the activator composition, the skin treatment system is an emulsion.

4. The skin treatment system of claim 1, wherein upon intermixing of the oxygenate composition and the activator composition, the skin treatment system is a gel.

5. The skin treatment system of claim 1, wherein upon intermixing of the oxygenate composition and the activator composition, the skin treatment system is a solution.

6. The skin treatment system of claim 1, wherein at least one of the oxygenate composition and the activator composition further includes at least one fatty compound.

7. The skin treatment system of claim 6, wherein the at least one fatty compound constitutes at least 30 wt % of the total weight of the skin treatment system.

8. The skin treatment system of claim 7, wherein the at least one fatty compound is selected from the group consisting of liquid petroleum jelly, polydecenes, fatty acids, liquid esters of fatty acids, fatty alcohols, and combinations thereof.

9. The skin treatment system of claim 1, wherein the at least one alkaline booster constitutes from 0.5 wt % to 5.5 wt % of the total weight of the skin treatment system.

10. The skin treatment system of claim 1, wherein the at least one alkaline booster is selected from the group consisting of organic amines and salts, mineral based salts, alkali metal hydroxides, alkali earth metal hydroxides, alkali metal salts, clays, and combinations thereof.

11. The skin treatment system of claim 10, wherein the at least one alkaline booster is selected from the group consisting of guanidine carbonate, arginine, monoethanolamine, triethanolamine, potassium hydroxide, sodium bicarbonate, sodium silicate, montmorillonite, and combinations thereof.

12. The skin treatment system of claim 1, wherein the at least one water-soluble peroxide decomposition catalyst constitutes from 0.1 wt % to 6 wt % of the total weight of the skin treatment system.

13. The skin treatment system of claim 1, wherein the at least one water-soluble peroxide decomposition catalyst is a metal salt.

14. The skin treatment system of claim 13, wherein the at least one water-soluble peroxide decomposition catalyst is selected from the group consisting of a manganese salt of pyrrolidone carboxylic acid, manganese gluconate, manganese sulfate, manganese chloride, a copper salt of pyrrolidone carboxylic acid, copper gluconate, copper sulfate, copper chloride, a zinc salt of pyrrolidone carboxylic acid, zinc gluconate, zinc sulfate, zinc chloride, and combinations thereof.

15. The skin treatment system of claim 1, wherein the at least one peroxide constitutes from 0.5 wt % to 5 wt % of the total weight of the skin treatment system.

16. The skin treatment system of claim 1, wherein the skin treatment system is free of ammonia and persulfate.

17. The skin treatment system of claim 1, wherein the skin treatment system is free of encapsulating microspheres.

18. The skin treatment system of claim 1, wherein the skin treatment system includes mechanical exfoliation agents.

19. The skin treatment system of claim 1, wherein the oxygenate composition and the activator composition are packaged in isolation from one another.

20. A skin treatment system, comprising:
an oxygenate composition including at least one peroxide in a first aqueous phase;
an activator composition including:
at least one alkaline booster in a second aqueous phase, the at least one alkaline booster being selected from the group consisting of guanidine carbonate, arginine, monoethanolamine, triethanolamine, potassium hydroxide, sodium bicarbonate, sodium silicate, montmorillonite, and combinations thereof; and
at least one water-soluble peroxide decomposition catalyst in the second aqueous phase, the at least one water-soluble peroxide decomposition catalyst being selected from the group consisting of a manganese salt of pyrrolidone carboxylic acid, manganese gluconate, manganese sulfate, manganese chloride, a copper salt of pyrrolidone carboxylic acid, copper gluconate, copper sulfate, copper chloride, a zinc salt of pyrrolidone carboxylic acid, zinc gluconate, zinc sulfate, zinc chloride, and combinations thereof; and
optionally, at least one fatty compound in at least one of the oxygenate composition and the activator composition, the at least one fatty compound being selected from the group consisting of liquid petroleum jelly, polydecenes, fatty acids, liquid esters of fatty acids, fatty alcohols, and combinations thereof,
wherein:
intermixing the oxygenate composition and the activator composition at least partially decomposes the at least one peroxide, liberating O₂;
the skin treatment system in contact with keratinous tissue induces reduced irritation to the keratinous tissue relative to a comparative skin treatment system which is identical to the skin treatment system except for lacking the at least one water-soluble peroxide decomposition catalyst;
intermixing the oxygenate composition and the activator composition produces reduced reactive oxygen species relative to the comparative skin treatment system;
intermixing the oxygenate composition and the activator composition liberates O₂ at an increased rate relative to the comparative skin treatment system;
upon intermixing of the oxygenate composition and the activator composition, the skin treatment system includes a form selected from the group consisting of an emulsion, a gel, a solution, and combinations thereof;
the optional at least one fatty compound constitutes at least 30 wt % of the total weight of the skin treatment system;
the at least one alkaline booster constitutes from 0.5 wt % to 5.5 wt % of the total weight of the skin treatment system;
the at least one water-soluble peroxide decomposition catalyst constitutes from 0.1 wt % to 6 wt % of the total weight of the skin treatment system;
the at least one peroxide constitutes from 0.5 wt % to 5 wt % of the total weight of the skin treatment system.
